(19) United States
(12) Patent Application Publication
(10) Pub. No.: US 2007/0032438 A1
(43) Pub. Date: Feb. 8, 2007

(54) Pharmaceutical compositions containing taxanes and methods for preparing the pharmaceutical compositions

(75) Inventors: Yu-Fang Hu, Chungli- City (TW); Yao-Kun Huang, Chungli City (TW); Shian-Bin Ye, Taipei (TW)

Correspondence Address: BACON & THOMAS, PLLC, 625 SLATERS LANE, FOURTH FLOOR, ALEXANDRIA, VA, 22314, US

(73) Assignee: TTY BIOPHARM COMPANY LIMITED, Taipei (TW)

(21) Appl. No.: 11/194,676

(22) Filed: Aug. 2, 2005

(51) Int. Cl. A61K 31/70; A61K 31/337; A61K 47/00

(52) U.S. Cl. 514/23; 514/449; 514/784

(57) ABSTRACT

Pharmaceutical compositions containing taxanes having about 5 to about 75% phospholipid, about 0.01 to about 50% of a surfactant of an HLB value greater than 10, about 0.01 to about 50% alcohol and about 0.01 to about 40% taxane(s) or a derivative thereof.

### BACKGROUND OF THE INVENTION

1. Field of Invention

The present invention is related to pharmaceutical compositions containing taxanes and methods for preparing the pharmaceutical compositions.

2. Description of the Related Art

Approval of the composition Paclitaxel™ was issued in December, 1992 by the FDA in the USA. Paclitaxel is a 1-P grade pharmaceutical formulation, “1” refers to a novel molecule and “P” refers to a formulation that is investigated firstly, so paclitaxel is demonstrated as being a potential drug for use in chemotherapy. The use and sale of paclitaxel has been an exciting achievement in the past years. Paclitaxel is as important as doxorubicin and cisplatin in the field of anti-cancer drugs.

Paclitaxel and docetaxel are taxoid drugs extracted from the bark of the Pacific yew tree (Taxus brevifolia) and the needles of the English yew tree, respectively. Both work against cancer by interfering with mitosis, but they each do it a little differently. However, the growth area of the Pacific yew tree is limited to the north west side of the United States and the west side of Canada. Often, two to six trees are required to prepare an appropriate pharmaceutical amount for a single patient. The tree grows slowly with a life span of about 30 to 100 years. Further, environmental protection groups raise objections to the felling of the Pacific yew trees. The above difficulties have delayed the onset of the commercial availability of the drug, resulting in concern about how to find a substitute for paclitaxel. Recently, it has reported that the Taxus Chinensis Rehd., which grows in China, also can achieve the same product as the aforementioned yew trees.

The development of isolating paclitaxel has been troublesome since the beginning of the research. After overcoming the problems of the source of paclitaxel, serious allergic reaction due to paclitaxel has been found during clinical trail phase I and this nearly resulted in termination of testing. The syndromes of the allergic reactions include dyspnea, hypotension, angioedema and systemic urticaria and the allergic reaction is similar with the reaction caused by the agents for angiography. Modification of the injection rate of the drug led to a lowering of the allergic reaction, and it was eventually found that the allergic reaction is caused by the solubilizers not by paclitaxel itself.

The solubility of paclitaxel is very low, so it is hardly soluble (less than 10 μg/ml), and this aspect also impedes the development of the drug. Paclitaxel is sold as Taxol® by Bristol-Myers Squibb and to date, Taxol® is the only issued taxoid drug. Taxol® contains 50% ethanol and 50% Cremophor EL as solubilizers. However, Cremophor EL is a nonionic surfactant and is a toxicant. Injecting a patient with Cremophor EL will cause serious allergic reaction, and it has been reported that Cremophor EL has even led to death. To prevent such side-effects, patients are pre-treated with corticosteroids, diphenhydramine (an antihistamine) or H2 blocker before they undergo paclitaxel therapy. After 30 minutes of the pretreatment, paclitaxel is given intravenously. Besides the serious hypersensitivity, how to prepare, how to administer taxanes, the stability of taxanes and the compatibility of taxanes still represent serious challenges to the medical workers. For example, paclitaxel cannot be kept inside a PVC container as toxicants will be dissolved from the PVC container, including drug containers, venous syringes, tubular filters and cannulae during injecting paclitaxel.

To increase the solubility of taxane in water, chemists synthesize docetaxel which can dissolve in water. However, docetaxel still cannot become a stable solution, and docetaxel needs the surfactant, Tween™, as a solvent. Therefore, the side effects caused by the surfactants still happen.

Obviously, the main problems of taxane injection drugs are due to the nonionic surfactants, so if the side effects caused by the surfactants can be solved, the patients will be treated without the pretreatment and this will promote the convenience for drug administration.

Liposomes containing taxane is the most well-known way for modifying the taxane injection drugs. However, taxane precipitates in only a few weeks. Furthermore, liposomes are milled into powder for storage, but the procedure for preparing liposomes and freeze drying method are complex and expensive with apparently no scope for cost reduction.

In other cases, taxanes are dissolved in dimethylacetamide (DMA), N-methylpyrrolidinone (NMP) to increase solubility of the taxanes. However, DMA and NMP both have toxicity and so are not widely applied.

In U.S. Pat. No. 6,096,331, taxanes are administered with a human albumin to obtain a nontoxic and soluble complex. In WO 02/26208, U.S. Pat. No. 5,616,330 and U.S. Pat. No. 6,348,491, taxanes are emulsified in the formulations. However, the cost is still high and the procedure is also complex. Further, taxanes precipitate easily in the emulsified formulations and cannot be stored stably.

Thus, a real need exists today for a taxane composition that is easy to solve and store stably.

### SUMMARY OF THE INVENTION

An aspect of the present invention is related to a pharmaceutical composition containing taxanes comprising about 5% (w/w) to 75% (w/w) phospholipid, about 0.05% (w/w) to 50% (w/w) a surfactant of an HLB value greater than 10, about 0.01 to 50% alcohol, and about 0.01 to 40% taxane(s) or a derivative thereof.

Preferably, said alcohol is selected from a group consisting of propylene glycol, polyethylene glycol, ethanol or more than one of the above alcohols, wherein the average molecular weight of polyethylene glycol is about 200 to 4000.

Preferably, the pharmaceutical composition comprises a condensed pharmaceutical composition.

Preferably, the pharmaceutical composition further comprises a diluent.

More preferably, said diluent is an injection.

More preferably, said injection is a glucose solution, a sodium chloride solution or Ringer's solution.

Most preferably, said diluent is Liposyn®, Soyacal®, Travemulsion® or Intralipid®.

Preferably, said surfactant is a phopholipid.

More preferably, said surfactant is Kollidon, puronic acid or lecithin.

Preferably, the concentration of said taxane(s) or a derivative thereof is in the range of from about 0.1 to about 2 mg/ml.

More preferably, the concentration of said taxane(s) or a derivative thereof is in the range of from about 0.16 to about 1 mg/ml.

Most preferably, the concentration of said taxane(s) or a derivative thereof is in the range of from about 0.16 to about 0.30 mg/ml.

Preferably, an emulsifying particle of said emulsifying agent is in the range of from 100 to 300 nm.

More preferably, an emulsifying particle of said emulsifying agent is in the range of from 150 to 200 nm.

Preferably, said taxanes is docetaxel or pacifiltaxel.

More preferably, said taxanes is docetaxel.

An aspect of the present invention is related to a method for preparing the pharmaceutical compositions characterizing in mixing all the following components well: about 5% (w/w) to 75% (w/w) phospholipid, about 0.05% (w/w) to 50% (w/w) a surfactant of an HLB value greater than 10, about 0.01 to 50% alcohol, and about 0.01 to 40% taxane(s) or a derivative thereof, wherein all components are mixed well.

Preferably, said alcohol is selected from a group consisting of propylene glycol, polyethylene glycol, ethanol or more than one of the above alcohols, wherein the average molecular weight of polyethylene glycol is about 200 to 4000.

Preferably, said taxane(s) or a derivative thereof is dissolved in an organic solvent and an emulsifying agent.

Other objectives, advantages and novel features of the invention will become more apparent from the following detailed description when taken in conjunction with the accompanying drawings.

### DETAILED DESCRIPTION OF THE INVENTION

The present invention is related to a taxane composition that does not contain Cremophor EL or Tween. The taxane composition in accordance with the present invention has a low toxicity and few side effects, further, the taxane composition in accordance with the present invention has a convenient way for its administration.

The term “taxanes” used herein referred to, includes but is not limited to, paclitaxel, docetaxel or derivative of taxanes. In the present invention, a pharmaceutical composition containing taxanes comprising pharmaceutically acceptable excipients without Tween. Preferably, the pharmaceutical composition is an injection; more preferably, the pharmaceutical composition in accordance with the present invention does not contain any water molecules. The pharmaceutical composition is dissolved in a solvent without containing water molecules, and can be administered without diluting beforehand. The pharmaceutical composition in accordance with the present invention is satisfactorily stable.

The process for preparing the pharmaceutical composition in accordance with the present invention requires only the mixing well of the components. The process described in the present invention needs fewer steps than the conventional one. Further, after emulsifying the pharmaceutical composition containing taxanes, the pharmaceutical composition becomes more stable. Due to the pharmaceutical composition not containing Tween, the cost and the side effects of the pharmaceutical composition of the present invention can be significantly reduced in relation to prior art.

Taxanes are only slightly soluble in water, but is well soluble in organic solutions including Cremophor EL, dimethylacetamide (DMA), dimethylsulfoxide (DMSO), ethanol, Tween, polyethylene glycol (PEG), propylene glycol (PG). However, taxanes will precipitate in the above solutions if they come into contact with water. Even when solved in nonionic surfactants, taxanes also become precipitated when diluting with an injection after 4 to 8 hours. Taxanes also can dissolve in oily solutions including oleic acid, safflower oils, corn oil, soybean oil.

The process for preparing the pharmaceutical composition containing taxanes comprises dissolving taxanes in an organic solution and an emulsifying agent without water, then mixing with a co-surfactant, such as Kollidon, puronic acid, lecithin or phospholipid. The pharmaceutical composition in accordance with the present invention is only mixed with an injection before use. Preferably, the pharmaceutical composition is an injection. After 24 hours of injection, taxanes in the emulsifying agent are still stable, and no crystals form. The sizes of the emulsifying particles containing taxanes in the pharmaceutical composition neither change nor cause creaming, and the emulsifying particles can prevent light that would degrade or damage taxanes.

The way to prepare the present invention is to only mix the components well by a conventional stirring apparatus and no complex apparatus is needed. Due to the present pharmaceutical composition not containing any water molecules, the present pharmaceutical composition can be stored for long periods. According to the stability test described in the present invention, the pharmaceutical composition can be stored at a room temperature for more than two years. In the present invention, taxanes may be dissolved in a condensed emulsifying agent and can be diluted and then administered easily.

The concentration of taxanes of the pharmaceutical composition may be 40 mg/ml, and can be diluted to 0.16 to 0.30 mg/ml for clinical injection. To administer the present pharmaceutical composition is the same as the conventional products such as Taxotere®, and has fewer side effects.

This invention is illustrated further by the following non-limiting examples. All of the literature and publications recited in the context of the present disclosure are incorporated herein by reference.

### EXAMPLES

### Example 1

The components to prepare the present pharmaceutical composition are described in Table 1.

TABLE 1
Components in a condensed docetaxel composition

| Components | Dose | |
|---|---|---|
| Doceltaxel | 4 | g |
| DSPC* | 10 | g |
| Lutrol | 45 | g |
| Kollidon | 2 | g |
| PEG 200 | 5 | g |
| Ethanol (q.s.) | 100 | ml |

*DSPC is an abbreviation for 1,2-diacyl-sn-glycerol-3-Phosphocholine.

The procedure for preparing the pharmaceutical composition is described as follows. The excipients including DSPC, Lutrol, Kollidon, PEG 200 and ethanol were mixed and then dissolved in a 40° C. water bath. When all the excipients had dissolved, taxanes were added into the above excipient liquid to obtain a mixture. The mixture was swung in a 40° C. water bath for 2 hours to obtain a condensed pharmaceutical composition containing taxanes. The pharmaceutical composition containing taxanes was stored at 25° C.±2° C. and 40° C.±2° C. for 6 months and no degradation was observed (Table 2).

TABLE 2
Stability test for a condensed pharmaceutical composition

Concentration (mg/ml)

| Time | 25° C. ± 2° C. | 40° C. ± 2° C. |
|---|---|---|
| Begin | 40.12 ± 0.22 | 40.32 ± 0.22 |
| 1^{st} month | 40.11 ± 0.15 | 40.28 ± 0.31 |
| 2^{nd} month | 40.01 ± 0.3.7 | 40.23 ± 0.25 |
| 4^{th} month | 40.22 ± 0.13 | 40.31 ± 0.29 |
| 6^{th} month | 40.21 ± 0.21 | 40.33 ± 0.23 |

2.5 ml of the condensed pharmaceutical composition was added to 500 ml 5% Dextrose® and shaken for 1 minute to mix the pharmaceutical composition well for the stability test and the crystal forming test. A filter with 0.45 μm sieve was used for filtering the pharmaceutical composition and to test the crystal forming rate. The above test demonstrated that no crystal formed in 24 hours and the results are shown in Table 3. Further, the emulsifying particles maintained sizes of 150 to 20 nm during the 24-hour period. In fact, the sizes of the emulsifying particles do not significantly change after the pharmaceutical composition has been mixed with 5% Dextrose®.

TABLE 3
Stability test for the pharmaceutical composition containing
taxanes

| | | | Particle size (nm*) |
|---|---|---|---|
| | | | of the present |
| | Concentration (mg/ml) | | pharmaceutical |
| Time | Before filtering | After filtering | composition |
| Begin | 1.71 ± 0.02 | 1.79 ± 0.04 | 152.8 ± 73.1 |
| 4 hour | NA | 1.74 ± 0.08 | 161.3 ± 61.8 |
| 8 hour | NA | 1.77 ± 0.12 | 166.1 ± 42.4 |
| 12 hour | 1.73 ± 0.04 | 1.72 ± 0.06 | 172.8 ± 51.3 |
| 18 hour | NA | 1.70 ± 0.09 | 155.4 ± 81.4 |
| 24 hour | 1.77 ± 0.07 | 1.72 ± 0.12 | 177.0 ± 58.4 |

*Test by laser scattering.

### Example 2

The present example for preparing the pharmaceutical composition containing taxanes, and phospholipid in the excipient can be any kind of phospholipid such as Centrophase 31. The components of the pharmaceutical composition described in example 2 are listed in Table 4.

TABLE 4
Components of the pharmaceutical composition containing
taxanes

| Component | Dose | |
|---|---|---|
| Docetaxel | 3 | grams |
| Phospholipid (Centrophase 31) | 20 | grams |
| Deoxycholic acid | 30 | grams |
| Ethanol g.s. | 100 | ml |

The procedure for preparing the pharmaceutical composition is described as follows. The excipients including Centrophase 31 and ethanol were mixed and then dissolved in a 40° C. water bath. When all the excipients had dissolved, taxanes were added into the above excipient liquid to obtain a mixture. The mixture was swung in a 40° C. water bath for 2 hours to obtain a condensed pharmaceutical composition containing taxanes. The pharmaceutical composition containing taxanes was stored at 25° C.±2° C. and 40° C.±2° C. for 6 months and no degradation was observed (Table 5).

TABLE 5
Stability test for a condensed pharmaceutical composition

Concentration (mg/ml)

| Time | 25° C. ± 2° C. | 40° C. ± 2° C. |
|---|---|---|
| Begin | 32.31 ± 0.22 | 32.31 ± 0.22 |
| 1^{st} month | 32.91 ± 0.35 | 32.60 ± 0.31 |
| 2^{nd} month | 33.11 ± 0.30 | 32.46 ± 0.25 |
| 4^{th} month | 32.44 ± 0.13 | 32.01 ± 0.29 |
| 6^{th} month | 32.48 ± 0.21 | 32.17 ± 0.23 |

5 ml of the condensed pharmaceutical composition was added to 500 ml 5% Dextrose® and shaken for 1 minute to mix the pharmaceutical composition well for the stability test and the crystal forming test. A filter with 0.45 μm sieve was used for filtering the pharmaceutical preparation and to test the crystal forming rate. The above test demonstrated that no crystal formed within 24 hours and the results are shown in Table 6. Further, the emulsifying particles maintained sizes of 150 to 20 nm during the 24-hour period.

TABLE 6
Stability test for the pharmaceutical composition containing
taxanes

| | Concentration (mg/ml) | |
|---|---|---|
| Time | Before filtering | After filtering |
| Begin | 0.28 ± 0.02 | 0.28 ± 0.02 |
| 4 hour | NA | 0.29 ± 0.03 |
| 8 hour | NA | 0.29 ± 0.02 |
| 12 hour | 0.29 ± 0.04 | 0.29 ± 0.03 |
| 18 hour | NA | 0.29 ± 0.02 |
| 24 hour | 0.29 ± 0.03 | 0.28 ± 0.04 |

* Test by laser scattering.

### Example 3

The present example for preparing the pharmaceutical composition containing taxanes, and the pharmaceutical composition has excellent stability. The components of the pharmaceutical composition described in example 3 are listed in Table 7.

TABLE 7
Components of the pharmaceutical composition containing
taxanes

| Component | Dose | |
|---|---|---|
| Docetaxel | 4 | grams |
| Phospholipon 90G | 30 | grams |
| Deoxycholic acid | 3 | grams |
| Ethanol | 70 | ml |

The procedure for preparing the pharmaceutical composition is described as follows. The excipients including Phospholipon 90G, and ethanol were mixed and then dissolved in a 40° C. water bath. When all the excipients had dissolved, taxanes were added into the above excipient liquid to obtain a mixture. The mixture was swung in a 40° C. water bath for 2 hours to obtain a condensed pharmaceutical composition containing taxanes. The pharmaceutical composition containing taxanes was stored at 25° C.±2° C. and 40° C.±2° C. for 6 months and no degradation was observed. (Table 8).

TABLE 8
Stability test for a condensed pharmaceutical composition

Concentration (mg/ml)

| Time | 25° C. ± 2° C. | 40° C. ± 2° C. |
|---|---|---|
| Begin | 40.15 ± 0.27 | 40.32 ± 0.16 |
| 1^{st} month | 40.01 ± 0.17 | 40.52 ± 0.12 |
| 2^{nd} month | 40.01 ± 0.25 | 40.21 ± 0.32 |
| 4^{th} month | 40.43 ± 0.58 | 40.37 ± 0.52 |
| 6^{th} month | 40.33 ± 0.21 | 40.15 ± 0.27 |

12.5 ml of the condensed pharmaceutical composition was added to 500 ml 5% Dextrose® and shaken for 1 minute to mix the pharmaceutical composition well for the stability test and the crystal forming test. A filter with 0.45 μm sieve was used for filtering the pharmaceutical composition and to test the crystal forming rate. The above test demonstrated that no crystal formed within 24 hours and the results are shown in Table 9. Further, the emulsifying particles maintained their sizes of 150 to 20 nm during the 24-hour period.

TABLE 9
Stability test for the pharmaceutical composition containing
taxanes

| | | | Particle size (nm*) |
|---|---|---|---|
| | | | of the present |
| | Concentration (mg/ml) | | pharmaceutical |
| Time | Before filtering | After filtering | composition |
| Begin | 1.08 ± 0.03 | 1.09 ± 0.02 | 176.2 ± 49.1 |
| 4 hour | NA | 1.08 ± 0.04 | 159.0 ± 59.7 |
| 8 hour | NA | 1.10 ± 0.03 | 171.5 ± 51.8 |
| 12 hour | 1.09 ± 0.03 | 1.08 ± 0.05 | 162.3 ± 43.1 |
| 18 hour | NA | 1.70 ± 0.09 | 155.4 ± 81.4 |
| 24 hour | 1.77 ± 0.07 | 1.72 ± 0.12 | 177.0 ± 58.4 |

*Test by laser scattering.

### Example 4

The present example for preparing the pharmaceutical composition containing taxanes, and the pharmaceutical composition with no enhancer (HLB>10) still had excellent stability. The components of the pharmaceutical composition described in example 4 are listed in Table 10.

TABLE 10
Components of the pharmaceutical composition containing
taxanes

| Component | Dose | |
|---|---|---|
| Docetaxel | 1.8 | grams |
| Phospholipon 90G | 22 | grams |
| Propylene glycol | 40 | grams |
| Ethanol | 100 | ml |

The procedure for preparing the pharmaceutical composition is described as follows. The excipients including Phospholipon 90G, propylene glycol and ethanol were mixed and then dissolved in a 40° C. water bath. When all the excipients had dissolved, taxanes were added into the above excipient liquid to obtain a mixture. The mixture was swung in a 40° C. water bath to obtain a condensed pharmaceutical composition containing taxanes. The pharmaceutical composition containing taxanes was stored at 25° C.±2° C. and 40° C.±2° C. for 6 months and no degradation was observed (Table 11).

TABLE 11
Stability test for a condensed pharmaceutical composition
(Table 10)

Concentration (mg/ml)

| Time | 25° C. ± 2° C. | 40° C. ± 2° C. |
|---|---|---|
| Begin | 18.15 ± 0.30 | 18.28 ± 0.30 |
| 1^{st} month | 18.01 ± 0.57 | 18.22 ± 0.31 |
| 2^{nd} month | 18.09 ± 0.33 | 18.13 ± 0.54 |
| 4^{th} month | 18.29 ± 0.34 | 18.25 ± 0.58 |
| 6^{th} month | 18.13 ± 0.69 | 18.16 ± 0.23 |

20 ml of the condensed pharmaceutical composition was added to into 500 ml 5% Dextrose® and shaken 1 minute to mix the pharmaceutical composition well for the stability test and the crystal forming test. A filter with 0.45 μm sieve was used for filtering the pharmaceutical composition and to test the crystal forming rate. The above test demonstrated that no crystal formed within 24 hours and the results are shown in Table 12.

TABLE 12
Stability test for the pharmaceutical composition containing
taxanes

Concentration (mg/ml)

| Time | Before filtering | After filtering |
|---|---|---|
| Begin | 0.73 ± 0.03 | 0.73 ± 0.06 |
| 4 hour | NA | 0.74 ± 0.04 |
| 8 hour | NA | 0.76 ± 0.04 |
| 12 hour | 0.74 ± 0.03 | 0.74 ± 0.06 |
| 18 hour | NA | 0.74 ± 0.07 |
| 24 hour | 0.74 ± 0.05 | 0.74 ± 0.07 |

*Test by laser scattering.

### Example 5

The present example is for preparing the pharmaceutical composition containing taxanes with excellent stability. The components of the pharmaceutical composition described in example 5 are listed in Table 13.

TABLE 13
Components of the pharmaceutical composition containing
taxanes

| Component | Dose |
|---|---|
| Docetaxel | 4 grams |
| Phospholipon 90G | 40 grams |
| Solutrol HS15 | 10 grams |
| Ethanol | 60 ml |

The procedure for preparing the pharmaceutical composition is described as follows. The excipients including Phospholipon 90G and Solutrol HS15 and docetaxel were mixed and dissolved in a water bath to obtain a condensed pharmaceutical composition containing taxanes. The pharmaceutical composition containing taxanes was stored at 25° C.±2° C. and 40° C.±2° C. for 6 months and no degradation was observed (Table 14).

TABLE 14
Stability test for a condensed pharmaceutical composition
(Table 13)

Concentration (mg/ml)

| Time | 25° C. ± 2° C. | 40° C. ± 2° C. |
|---|---|---|
| Begin | 39.45 ± 0.29 | 39.55 ± 0.42 |
| 1^{st} month | 39.50 ± 0.51 | 39.59 ± 0.37 |
| 2^{nd} month | 39.43 ± 0.23 | 39.42 ± 0.51 |
| 4^{th} month | 39.52 ± 0.34 | 39.54 ± 0.49 |
| 6^{th} month | 39.61 ± 0.65 | 39.43 ± 0.33 |

3.75 ml of the condensed pharmaceutical composition was added to into 500 ml 5% Dextrose® and shaken for 1 minute to mix the pharmaceutical composition well for the stability test and the crystal forming test. A filter with 0.45 μm sieve was used for filtering the pharmaceutical and to test the crystal forming rate. The above test demonstrated that no crystal formed within 24 hours and the results are shown in Table 15. Further, the emulsifying particles maintained sizes of 150 to 20 nm during the 24-hour period.

TABLE 15
Stability test for the pharmaceutical composition containing
taxanes

| | | | Particle size (nm*) |
|---|---|---|---|
| | | | of the present |
| | Concentration (mg/ml) | | pharmaceutical |
| Time | Before filtering | After filtering | composition |
| Begin | 0.31 ± 0.02 | 0.31 ± 0.06 | 173.5 ± 53.9 |
| 4 hour | NA | 0.32 ± 0.02 | 179.1 ± 39.1 |
| 8 hour | NA | 0.30 ± 0.04 | 163.5 ± 38.5 |
| 12 hour | 0.32 ± 0.03 | 0.31 ± 0.04 | 158.9 ± 57.2 |
| 18 hour | NA | 0.31 ± 0.07 | 165.7 ± 42.8 |
| 24 hour | 0.31 ± 0.04 | 0.31 ± 0.05 | 182.4 ± 46.8 |

*Test by laser scattering.

Even though numerous characteristics and advantages of the present invention have been set forth in the foregoing description, together with details of the structure and features of the invention, the disclosure is illustrative only. Changes may be made in the details, especially in matters of shape, size, and arrangement of parts within the principles of the invention to the full extent indicated by the broad general meaning of the terms in which the appended claims are expressed.

What is claimed is:

1. A pharmaceutical composition containing taxanes comprising
about 5% (w/w) to 75% (w/w) phospholipid,
about 0.05% (w/w) to 50% (w/w) of a surfactant of an HLB value greater than 10,
about 0.01 to 50% alcohol, and
about 0.01 to 40% taxane(s) or a derivative thereof.

2. The pharmaceutical composition as claimed in claim 1, wherein said alcohol is selected from a group consisting of propylene glycol, polyethylene glycol, ethanol and more than one of the above alcohols, wherein the average molecular weight of polyethylene glycol is about 200 to 4000.

3. The pharmaceutical composition as claimed in claim 1 where the pharmaceutical composition is a condensed pharmaceutical composition.

4. The pharmaceutical composition as claimed in claim 1 further comprising a diluent.

5. The pharmaceutical composition as claimed in claim 4, wherein said diluent is an injection.

6. The pharmaceutical composition as claimed in claim 5, wherein said injection is a glucose solution, a sodium chloride solution or Ringer's solution.

7. The pharmaceutical composition as claimed in claim 1, wherein said surfactant is a phopholipid.

8. The pharmaceutical composition as claimed in claim 7, wherein said surfactant is Kollidon, puronic acid or lecithin.

9. The pharmaceutical composition as claimed in claim 4, wherein the concentration of said taxane(s) or a derivative thereof is about 0.1 to about 2 mg/ml.

10. The pharmaceutical composition as claimed in claim 9, wherein the concentration of said taxane(s) or a derivative thereof is about 0.16 to about 1 mg/ml.

11. The pharmaceutical composition as claimed in claim 10, wherein the concentration of said taxane(s) or a derivative thereof is about 0.16 to about 0.30 mg/ml.

12. The pharmaceutical composition as claimed in claim 3, wherein an emulsifying particle of said emulsifying agent is in the range of from 100 to 300 nm.

13. The pharmaceutical composition as claimed in claim 12, wherein an emulsifying particle of said emulsifying agent is in the range of from 150 to 200 nm.

14. The pharmaceutical composition as claimed in claim 1, wherein said taxane is docetaxel or pacifiltaxel.

15. The pharmaceutical composition as claimed in claim 14, wherein said taxane is docetaxel.

16. The pharmaceutical composition as claimed in claim 5, wherein the concentration of said taxane(s) or a derivative thereof is about 0.1 to about 2 mg/ml.

17. The pharmaceutical composition as claimed in claim 6, wherein the concentration of said taxane(s) or a derivative thereof is about 0.1 to about 2 mg/ml.

18. A method for preparing pharmaceutical compositions characterized in mixing all following components:
about 5% (w/w) to about 75% (w/w) phospholipid,
about 0.05% (w/w) to about 50% (w/w) of a surfactant of an HLB value greater than 10,
about 0.01 to about 50% alcohol, and
about 0.01 to about 40% taxane(s) or a derivative thereof.

19. The method as claimed in claim 18, wherein said alcohol is selected from a group consisting of propylene glycol, polyethylene glycol, ethanol and more than one of the above alcohols, wherein the average molecular weight of polyethylene glycol is about 200 to 4000.

20. The method as claimed in claim 18, wherein said taxane(s) or a derivative thereof is dissolved in an organic solvent and an emulsifying agent.
